(19) United States
(12) Patent Application Publication
(10) Pub. No.: US 2023/0129143 A1
(43) Pub. Date: Apr. 27, 2023

(54) CATALYST FOR THE HYDROGENATION OF AROMATIC COMPOUNDS OBTAINED FROM MELTED SALTS AND AN ORGANIC ADDITIVE

(71) Applicant: IFP Energies nouvelles, Rueil-Malmaison Cedex (FR)

(72) Inventors: Malika BOUALLEG, Rueil-Malmaison Cedex (FR); Laetitia JOTHIE, Rueil-Malmaison Cedex (FR)

(73) Assignee: IFP Energies nouvelles, Rueil-Malmaison Cedex (FR)

(21) Appl. No.: 17/784,244

(22) PCT Filed: Dec. 4, 2020
(86) PCT No.: PCT/EP2020/084661

§ 371 Date: Jun. 10, 2022

(30) Foreign Application Priority Data
Dec. 17, 2019 (FR) 1914602

(51) Int. Cl. B01J 37/08; B01J 21/04; B01J 23/755; B01J 35/00; B01J 35/10; B01J 37/02; C07C 5/10

(52) U.S. Cl. CPC B01J 37/088; B01J 21/04; B01J 23/755; B01J 35/0013; B01J 35/1052; B01J 37/0203; C07C 5/10; C07C 2521/04; C07C 2523/76; B01J 2231/641

(57) ABSTRACT

Catalyst for the hydrogenation of aromatic compounds capable of being obtained by the process comprising at least the following stages:
a) the alumina support is brought into contact with at least one organic additive;
b) the alumina support is brought into contact with at least one nickel metal salt, the melting point of said metal salt of which is between 20° C. and 150° C.;
c) the solid mixture obtained on conclusion of stages a) and b) is heated with stirring;
d) the catalyst precursor obtained on conclusion of stage c) is dried;
e) a stage of heat treatment of the dried catalyst precursor obtained on conclusion of stage d) is carried out.

### TECHNICAL FIELD

The present invention relates to a catalyst intended particularly for the hydrogenation of unsaturated hydrocarbons and more particularly in the hydrogenation of aromatic compounds.

### STATE OF THE ART

The most active catalysts in hydrogenation reactions are conventionally based on noble metals, such as palladium or platinum. These catalysts are used industrially in refining and in petrochemistry for the purification of certain petroleum fractions by hydrogenation, in particular in reactions for the selective hydrogenation of polyunsaturated molecules, such as diolefins, acetylenics or alkenylaromatics, or in reactions for the hydrogenation of aromatics. It is often proposed to replace palladium with nickel, a metal which is less active than palladium, and which it is therefore necessary to have in a larger amount in the catalyst. Thus, nickel-based catalysts generally have a metal content of between 5% and 60% by weight of nickel, with respect to the catalyst.

The rate of the hydrogenation reaction is governed by several criteria, such as the diffusion of the reactants at the surface of the catalyst (external diffusional limitations), the diffusion of the reactants in the porosity of the support toward the active sites (internal diffusional limitations) and the intrinsic properties of the active phase, such as the size of the metal particles and the distribution of the active phase within the support.

As regards the internal diffusional limitations, it is important for the pore distribution of the macropores and mesopores to be appropriate for the desired reaction in order to provide for the diffusion of the reactants in the porosity of the support toward the active sites and also for the diffusion of the products formed toward the outside.

The catalysts for the hydrogenation of aromatic compounds are generally based on metals from Group VIII of the Periodic Table, preferably palladium or nickel. The metal is provided in the form of metal particles deposited on a support. The metal content, the size of the metal particles and the distribution of the active phase in the support are among the criteria which have an influence on the activity and the selectivity of the catalysts. As regards the size of the metal particles, it is generally accepted that the catalyst becomes more active as the size of the metal particles decreases. Furthermore, it is important to obtain a size distribution of the particles which is centered on the optimum value and also a narrow distribution around this value.

The often high content of nickel in the hydrogenation catalysts requires specific synthesis routes.

The most conventional route for the preparation of these catalysts is the impregnation of the support with an aqueous solution of a nickel precursor, generally followed by a drying and a calcination. Before they are used in hydrogenation reactions, these catalysts are generally reduced in order to obtain the active phase, which is in the metallic form (that is to say, in the zero valency state). Catalysts based on nickel on alumina prepared by just one impregnation stage generally make it possible to achieve nickel contents of between 12% and 15% by weight of nickel approximately, depending on the pore volume of the alumina used. If it is desired to prepare catalysts having a higher nickel content, several successive impregnations are often necessary in order to obtain the desired nickel content, followed by at least one drying stage and then optionally by a calcination stage between each impregnation.

Thus, the document WO2011/080515 describes a catalyst based on nickel on active alumina in hydrogenation, in particular of aromatics, the said catalyst having a nickel content of greater than 35% by weight, with respect to the total weight of the catalyst, and a high dispersion of the metallic nickel over the surface of an alumina having a very open porosity and having a high specific surface. The catalyst is prepared by at least four successive impregnations. The preparation of nickel catalysts having a high nickel content by the impregnation route thus implies a sequence of numerous stages, which increases the associated manufacturing costs.

Another preparation route also used to obtain catalysts having a high nickel content is coprecipitation. The coprecipitation generally consists in simultaneously running both an aluminum salt (for example aluminum nitrate) and a nickel salt (for example nickel nitrate) into a batch reactor. The two salts precipitate simultaneously. A high-temperature calcination is then necessary to bring about the transition of the alumina gel (for example boehmite) to alumina. Contents of up to 70% by weight of nickel are achieved by this preparation route. Catalysts prepared by coprecipitation are, for example, described in the documents U.S. Pat. Nos. 4,273,680, 8,518,851 and US 2010/0116717.

Finally, the route of preparation by cokneading is also known. Cokneading generally consists in mixing a nickel salt with an alumina gel, such as boehmite, the mixture produced being subsequently shaped, generally by extrusion, then dried and calcined. The document U.S. Pat. No. 5,478,791 describes a catalyst based on nickel on alumina having a nickel content of between 10% and 60% by weight and a size of nickel particles of between 15 and 60 nm, prepared by cokneading a nickel compound with an alumina gel, followed by a shaping, a drying and a reduction.

Furthermore, for the purpose of obtaining better catalytic performance qualities, in particular a better selectivity and/or activity, it is known in the state of the art to proceed to the use of additives of organic compounds type in the preparation of metal selective hydrogenation catalysts or metal catalysts for the hydrogenation of aromatics.

For example, the application FR 2 984 761 discloses a process for the preparation of a selective hydrogenation catalyst comprising a support and an active phase comprising a metal from Group VIII, said catalyst being prepared by a process comprising a stage of impregnation with a solution containing a precursor of the metal from Group VIII and an organic additive, more particularly an organic compound exhibiting from one to three carboxylic acid functional groups, a stage of drying the impregnated support and a stage of calcination of the dried support in order to obtain the catalyst.

The document US 2006/0149097 discloses a process for the hydrogenation of aromatic compounds of benzenepolycarboxylic acid type in the presence of a catalyst comprising an active phase comprising at least one metal from Group VIII, which catalyst is prepared by a process comprising a stage of impregnation with a solution containing a precursor of the metal from Group VIII and a stage of impregnation with an organic additive of amine or amino acid type. The stage of impregnation with the organic additive can be carried out before or after the stage of impregnation with the active phase, or even simultaneously.

Furthermore, the use of molten salts as precursors of the active phase of a catalyst or of a capture body is also known from the literature.

For example, the document U.S. Pat. No. 5,036,032 discloses a method for the preparation of a cobalt-based supported catalyst by bringing a support in contact (of the order of a few tens of seconds) in a bath of molten cobalt nitrate salt, followed by a stage of drying and of reduction without intermediate calcination. This method makes possible the preferential localization of the cobalt phase at the periphery of the support. However, the method does not make possible precise control of the amount of active phase (in this instance cobalt) deposited due to the very short contact time. Moreover, the absence of a calcination stage is risky since the reaction between the reduction element and the nitrates in the solid is highly exothermic. Finally, this method makes it necessary to handle large amounts of (toxic) cobalt nitrate in liquid form and at temperature, with ratios of approximately 4 grams of active-phase precursors per 1 gram of support. The catalysts obtained by this preparation route are used for the Fischer-Tropsch synthesis of hydrocarbons.

It is known, from Chem. Mater., 1999, 11, pp. 1999-2007, to prepare mixed phosphates via a route of molten salts type. The reaction mixture contains a metal precursor salt (in particular Ni(NO₃)₂ or Co(NO₃)₂), a source of phosphorus (NH₄H₂PO₄) and an alkali metal (Na or K) nitrate. These preparations are carried out at high temperatures of the order of 400° C. to 450° C. Solids of mixed phosphates type are obtained, for example Na₃Ni₂(P₂O₇)PO₄, K₂Ni₄(PO₄)₂P₂O₇ or Na₉CO₃(PO₄)₅. These solids can find applications in ion exchange, in high-temperature ion conduction or in catalysis.

The document GB 191308864 discloses a process for the synthesis of a bulk catalyst based on nickel or on cobalt for the production of hydrogen by steam reforming. These catalysts can be obtained by liquefaction of metal salts at moderate temperatures, then poured into a mold before calcination heat treatment.

The publication by J.-Y. Tilquin entitled “Intercalation of CoCl₂ into Graphite: Mixing Method vs Molten Salt Method”, published in Carbon, 35(2), pp. 299-306, 1997, proposes the use, in molten salt form, of a CoCl₂—NaCl mixture at high temperature (450-580° C.) for intercalation between graphite sheets. These graphite intercalation compounds find applications in catalysis for the reduction of oxygen in polymer electrolyte fuel cells.

Subject Matters of the Invention

The present invention thus relates to a new type of catalyst which, due to its specific process of preparation, makes it possible to obtain a catalyst comprising performance qualities at least as good, indeed even better, in terms of activity in the context of reactions for the hydrogenation of aromatic compounds, while using an amount of nickel-based active phase equal to, indeed even less than, that typically used in the state of the art. In addition, this preparation process results in a catalyst exhibiting a size of nickel particles of less than 18 nm, conferring a high intrinsic activity of the nickel active phase. This preparation process employed here makes it possible, without addition of solvent, and thus in a very limited number of stages and above all a lower number than the conventional preparation process (by impregnation), to obtain a catalyst, the catalytic performance qualities of which are superior to conventional catalysts (in particular no upstream preparation of solution with Ni and/or additive, and no intermediate drying).

A subject matter according to the invention relates to a catalyst for the hydrogenation of aromatic or polyaromatic compounds comprising a nickel-based active phase and an alumina support, said active phase does not comprising a metal from Group VIB, said catalyst comprising between 20% and 60% by weight of elemental nickel, with respect to the total weight of the catalyst, the size of the nickel particles in the catalyst, measured in oxide form, being less than 18 nm, said catalyst being capable of being obtained by the process comprising at least the following stages:
a) the alumina support is brought into contact with at least one organic additive comprising oxygen and/or nitrogen, the molar ratio of the organic additive to the nickel being greater than 0.05 mol/mol;
b) the alumina support is brought into contact with at least one nickel metal salt, at a temperature of less than the melting point of said nickel metal salt, in order to form a solid mixture, the ratio by weight of said metal salt to the alumina support being between 0.1 and 2.3, stages a) and b) being carried out either successively in this order, or simultaneously;
c) the solid mixture obtained on conclusion of stages a) and b) is heated with stirring to a temperature between the melting point of said metal salt and 200° C., in order to obtain a catalyst precursor;
d) the catalyst precursor obtained on conclusion of stage c) is dried at a temperature of less than 250° C. in order to obtain a dried catalyst precursor;
e) a stage of heat treatment of the dried catalyst precursor obtained on conclusion of stage d) is carried out at a temperature of between 250 and 1000° C.

Preferably, the size of the nickel particles in the catalyst, measured in oxide form, is between 0.5 and 12 nm, more preferentially between 1 and 5 nm.

Another subject matter according to the invention relates to a process for the preparation of a catalyst for the hydrogenation of aromatic or polyaromatic compounds comprising a nickel-based active phase and an alumina support, said active phase does not comprising a metal from Group VIB, said catalyst comprising between 20% and 60% by weight of elemental nickel, with respect to the total weight of the catalyst, the size of the nickel particles in the catalyst, measured in oxide form, being less than 18 nm, said process comprising the following stages:
a) the alumina support is brought into contact with at least one organic additive comprising oxygen and/or nitrogen, the molar ratio of the organic additive to the nickel being greater than 0.05 mol/mol;
b) the alumina support is brought into contact with at least one nickel metal salt, at a temperature of less than the melting point of said nickel metal salt, in order to form a solid mixture, the ratio by weight of said metal salt to the alumina support being between 0.1 and 2.3, stages a) and b) being carried out either successively in this order, or simultaneously;
c) the solid mixture obtained on conclusion of stages a) and b) is heated with stirring to a temperature between the melting point of said metal salt and 200° C., in order to obtain a catalyst precursor;
d) the catalyst precursor obtained on conclusion of stage c) is dried at a temperature of less than 250° C. in order to obtain a dried catalyst precursor;
e) a stage of heat treatment of the dried catalyst precursor obtained on conclusion of stage d) is carried out at a temperature of between 250 and 1000° C.

Preferably, the melting point of said metal salt is between 20° C. and 150° C.

Preferably, a stage e) of heat treatment of the dried catalyst precursor obtained in stage d) is carried out at a temperature of between 250° C. and 1000° C.

Preferably, the molar ratio of said organic additive introduced in stage a) to the element nickel introduced in stage b) is between 0.1 and 5.0 mol/mol.

In one embodiment according to the invention, stages a) and b) are carried out simultaneously.

Preferably, the organic additive is chosen from aldehydes including from 1 to 14 carbon atoms per molecule, ketones or polyketones including from 3 to 18 carbon atoms per molecule, ethers and esters including from 2 to 14 carbon atoms per molecule, alcohols or polyalcohols including from 1 to 14 carbon atoms per molecule and carboxylic acids or polycarboxylic acids including from 1 to 14 carbon atoms per molecule, or a combination of the various functional groups above.

More preferentially, said organic additive of stage a) is chosen from formic acid, formaldehyde, acetic acid, citric acid, oxalic acid, glycolic acid, malonic acid, levulinic acid, ethanol, methanol, ethyl formate, methyl formate, paraldehyde, acetaldehyde, γ-valerolactone, glucose and sorbitol.

More preferentially, the organic additive is chosen from citric acid, formic acid, glycolic acid, levulinic acid and oxalic acid.

Preferably, stage c) is carried out by means of a pan operating at a speed of between 4 and 70 revolutions per minute.

Preferably, in stage b), the ratio by weight of said metal salt to the alumina support is between 0.2 and 2.

Another subject matter according to the invention relates to a process for the hydrogenation of at least one aromatic or polyaromatic compound present in a hydrocarbon feedstock having a final boiling point of less than or equal to 650° C., said process being carried out in the gas phase or in the liquid phase, at a temperature of between 30 and 350° C., at a pressure of between 0.1 and 20 MPa, at a hydrogen/(aromatic compounds to be hydrogenated) molar ratio between 0.1 and 10 and at an hourly space velocity HSV of between 0.05 and 50 h⁻¹, in the presence of a catalyst according to the invention or prepared according to the preparation process according to the invention.

### DETAILED DESCRIPTION OF THE INVENTION

### Definitions

Subsequently, the groups of chemical elements are given according to the CAS classification (CRC Handbook of Chemistry and Physics, published by CRC Press, editor-in-chief D. R. Lide, 81st edition, 2000-2001). For example, Group VIII according to the CAS classification corresponds to the metals of Columns 8, 9 and 10 according to the new IUPAC classification.

The specific surface of the catalyst or of the support used for the preparation of the catalyst according to the invention is understood to mean the BET specific surface determined by nitrogen adsorption in accordance with the standard ASTM D 3663-78 drawn up from the Brunauer-Emmett-Teller method described in the journal “The Journal of the American Chemical Society”, 60, 309 (1938).

In the present patent application, the term “to comprise” is synonymous with (means the same thing as) “to include” and “to contain”, and is inclusive or open and does not exclude other elements not stated. It is understood that the term “to comprise” includes the exclusive and closed term “to consist”.

The term “macropores” is understood to mean pores, the opening of which is greater than 50 nm.

The term “mesopores” is understood to mean pores, the opening of which is between 2 nm and 50 nm, limits inclusive.

The term “micropores” is understood to mean pores, the opening of which is less than 2 nm.

Total pore volume of the catalyst or of the support used for the preparation of the catalyst according to the invention is understood to mean the volume measured by mercury intrusion porosimetry according to the standard ASTM D4284-83 at a maximum pressure of 4000 bar (400 MPa), using a surface tension of 484 dyne/cm and a contact angle of 140°. The wetting angle was taken equal to 140° following the recommendations of the publication “Techniques de l'ingénieur, traité analyse et caractérisation” [Techniques of the Engineer, Analysis and Characterization Treatise], pages 1050-1055, written by Jean Charpin and Bernard Rasneur.

In order to obtain better accuracy, the value of the total pore volume corresponds to the value of the total pore volume measured by mercury intrusion porosimetry measured on the sample minus the value of the total pore volume measured by mercury intrusion porosimetry measured on the same sample for a pressure corresponding to 30 psi (approximately 0.2 MPa).

The volume of the macropores and of the mesopores is measured by mercury intrusion porosimetry according to the standard ASTM D4284-83 at a maximum pressure of 4000 bar (400 MPa), using a surface tension of 484 dyne/cm and a contact angle of 140°. The value from which the mercury fills all the intergranular voids is set at 0.2 MPa and it is considered that, above this, the mercury penetrates into the pores of the sample.

The macropore volume of the catalyst or of the support used for the preparation of the catalyst according to the invention is defined as being the cumulative volume of mercury introduced at a pressure of between 0.2 MPa and 30 MPa, corresponding to the volume present in the pores with an apparent diameter of greater than 50 nm.

The mesopore volume of the catalyst or of the support used for the preparation of the catalyst according to the invention is defined as being the cumulative volume of mercury introduced at a pressure of between 30 MPa and 400 MPa, corresponding to the volume present in the pores with an apparent diameter of between 2 and 50 nm.

The volume of the micropores is measured by nitrogen porosimetry. The quantitative analysis of the microporosity is carried out starting from the “t” method (method of Lippens-De Boer, 1965), which corresponds to a transform of the starting adsorption isotherm, as described in the work “Adsorption by Powders and Porous Solids. Principles, Methodology and Applications”, written by F. Rouquérol, J. Rouquérol and K. Sing, Academic Press, 1999.

The median mesopore diameter is also defined as being the diameter such that all the pores, among the combined pores constituting the mesopore volume, with a size of less than this diameter constitute 50% of the total mesopore volume determined by mercury porosimetry intrusion.

The median macropore diameter is also defined as being the diameter such that all the pores, among the combined pores constituting the macropore volume, with a size of less than this diameter constitute 50% of the total macropore volume determined by mercury porosimetry intrusion.

The term “size of the nickel particles” is understood to mean the diameter of the nickel crystallites in oxide form. The diameter of the nickel crystallites in oxide form is determined by X-ray diffraction, from the width of the diffraction line located at the angle 20=43° (that is to say, along the crystallographic direction [200]) using the Scherrer relationship. This method, used in X-ray diffraction on polycrystalline samples or powders, which links the full width at half maximum of the diffraction peaks to the size of the particles, is described in detail in the reference: Appl. Cryst. (1978), 11, 102-113, “Scherrer after sixty years: A survey and some new results in the determination of crystallite size”, J. I. Langford and A. J. C. Wilson.

The nickel content is measured by X-ray fluorescence.

Catalyst

The nickel content in said catalyst according to the invention is advantageously between 20% and 60% by weight of element nickel, with respect to the total weight of the catalyst, more preferentially between 20% and 50% by weight and more preferentially still between 20% and 45% by weight, with respect to the total weight of the catalyst.

The active phase of the catalyst does not comprise a metal from Group VIB. In particular, it does not comprise molybdenum or tungsten. Preferably, the catalyst consists of an active phase consisting solely of nickel and of an alumina support.

The size of the nickel particles in the catalyst, measured in oxide form, is less than 18 nm, preferably less than 15 nm, more preferentially between 0.5 and 12 nm, in a preferred way between 1 and 8 nm, in an even more preferred way between 1 and 6 nm and more preferentially still between 1 and 5 nm.

Said catalyst is generally presented in all the forms known to a person skilled in the art, for example in the form of beads (generally having a diameter of between 1 and 8 mm), of extrudates, of blocks or of hollow cylinders. Preferably, it consists of extrudates with a diameter generally of between 0.5 and 10 mm, preferably between 0.8 and 3.2 mm and very preferably between 1.0 and 2.5 mm and with a mean length of between 0.5 and 20 mm. The term “mean diameter” of the extrudates is understood to mean the mean diameter of the circle circumscribed in the cross section of these extrudates. The catalyst can advantageously be presented in the form of cylindrical, multilobal, trilobal or quadrilobal extrudates. Preferably, its form will be trilobal or quadrilobal. The shape of the lobes will be able to be adjusted according to all the known methods of the prior art.

The specific surface of the catalyst is generally greater than or equal to 30 m²/g, preferably greater than or equal to 50 m²/g, more preferentially between 60 m²/g and 500 m²/g and more preferentially still between 70 m²/g and 400 m²/g.

The total pore volume of the catalyst is generally between 0.1 and 1.5 cm³/g, preferably between 0.35 and 1.2 cm³/g, and more preferentially still between 0.4 and 1.0 cm³/g, and more preferentially still between 0.45 and 0.9 cm³/g.

The catalyst advantageously exhibits a macropore volume of less than or equal to 0.6 ml/g, preferably of less than or equal to 0.5 ml/g, more preferentially of less than or equal to 0.4 ml/g and more preferentially still of less than or equal to 0.3 ml/g.

The mesopore volume of the catalyst is generally at least 0.10 ml/g, preferably at least 0.20 ml/g, in a preferred way between 0.25 ml/g and 0.80 ml/g and in a more preferred way between 0.30 and 0.65 ml/g.

The median mesopore diameter of the catalyst is advantageously between 3 and 25 nm, preferably between 6 and 20 nm and particularly preferably between 8 and 18 nm.

The catalyst advantageously exhibits a median macropore diameter of between 50 and 1500 nm, preferably between 80 and 1000 nm and more preferably still of between 250 and 800 nm.

Preferably, the catalyst exhibits a low microporosity; very preferably, it does not exhibit any microporosity.

Support

According to the invention, the support is an alumina, that is to say that the support comprises at least 95%, preferably at least 98% and particularly preferably at least 99% by weight of alumina, with respect to the weight of the support. The alumina generally exhibits a crystallographic structure of the δ-, γ- or θ-alumina type, alone or as a mixture.

According to the invention, the alumina support can comprise impurities such as oxides of metals from Groups IIA, IIIB, IVB, IIB, IIIA and IVA according to the CAS classification, preferably silica, titanium dioxide, zirconium dioxide, zinc oxide, magnesium oxide and calcium oxide, or also alkali metals, preferably lithium, sodium or potassium, and/or alkaline earth metals, preferably magnesium, calcium, strontium or barium, or also sulfur.

The specific surface of the support is generally greater than or equal to 30 m²/g, preferably greater than or equal to 50 m²/g, more preferentially between 60 m²/g and 500 m²/g and more preferentially still between 70 m²/g and 400 m²/g.

The total pore volume of the support is generally between 0.1 and 1.5 cm³/g, preferably between 0.35 and 1.2 cm³/g, and more preferentially still between 0.4 and 1.0 cm³/g, and more preferentially still between 0.45 and 0.9 cm³/g.

The support advantageously exhibits a macropore volume of less than or equal to 0.6 ml/g, preferably of less than or equal to 0.5 ml/g, more preferentially of less than or equal to 0.4 ml/g and more preferentially still of less than or equal to 0.3 ml/g.

The mesopore volume of the support is generally at least 0.10 ml/g, preferably at least 0.20 ml/g, in a preferred way between 0.25 ml/g and 0.80 ml/g and in a more preferred way between 0.30 and 0.65 ml/g.

The median mesopore diameter of the support is advantageously between 3 and 25 nm, preferably between 6 and 20 nm and particularly preferably between 8 and 18 nm.

The support advantageously exhibits a median macropore diameter of between 50 and 1500 nm, preferably between 80 and 1000 nm and more preferably still of between 250 and 800 nm.

Preferably, the support exhibits a low microporosity; very preferably, it does not exhibit any microporosity.

Preparation Process

The stages of the process for the preparation of the catalyst are described in detail below.

Stage a)

According to stage a) of the process for the preparation of the catalyst, the support is brought into contact with at least at least one organic additive comprising oxygen and/or nitrogen, preferably chosen from aldehydes including from 1 to 14 carbon atoms per molecule (preferably from 2 to 12), ketones or polyketones including from 3 to 18 (preferably from 3 to 12) carbon atoms per molecule, ethers or esters including from 2 to 14 (preferably from 3 to 12) carbon atoms per molecule, alcohols or polyalcohols including from 1 to 14 (preferably from 2 to 12) carbon atoms per molecule and carboxylic acids or polycarboxylic acids including from 1 to 14 (preferably from 1 to 12) carbon atoms per molecule. The organic additive can be composed of a combination of the various functional groups mentioned above.

Preferably, the organic additive is chosen from formic acid HCOOH, formaldehyde CH₂O, acetic acid CH₃COOH, citric acid, oxalic acid, glycolic acid (HOOC—CH₂—OH), malonic acid (HOOC—CH₂—COOH), levulinic acid (CH₃CCH₂CH₂CO₂H), ethanol, methanol, ethyl formate HCOOC₂H₅, methyl formate HCOOCH₃, paraldehyde (CH₃—CHO)₃, acetaldehyde C₂H₄O, γ-valerolactone (C₅H₈O₂), glucose and sorbitol.

Particularly preferably, the organic additive is chosen from citric acid, formic acid, glycolic acid, levulinic acid and oxalic acid.

In one embodiment according to the invention, said stage a) is carried out by bringing the support into contact with at least one organic additive in the form of a powder.

In another embodiment according to the invention, said stage a) is carried out by bringing the support into contact with at least one organic additive in the form of a powder dissolved in a minimum amount of water. Minimum amount of water is understood to mean the amount of water making possible the at least partial dissolution of said organic additive in the water. This minimum amount of water may not be comparable to a solvent. In this case, and when the stage of introduction of the additive is carried out separately from the introduction of the precursor of the active phase of the catalyst (i.e. stages a) and b) are carried out separately), each stage of bringing the support into contact with the organic additive is advantageously followed by drying at a temperature of less than 250° C., preferably between 15 and 240° C., more preferentially between 30 and 220° C.

The contacting operation is generally carried out at a temperature between 0 and 70° C., preferably between 10 and 60° C. and particularly preferably at ambient temperature.

According to stage a), the operation of bringing said porous support and the organic additive into contact can be carried out by any method known to a person skilled in the art. Preferably, use may be made of convective mixers, drum mixers or static mixers. Stage a) is advantageously carried out for a period of time of between 5 minutes and 5 hours, depending on the type of mixer used, preferably between 10 minutes and 4 hours.

According to the invention, the molar ratio of the organic additive to the nickel is greater than 0.05 mol/mol, preferably between 0.1 and 5 mol/mol, more preferentially between 0.12 and 3 mol/mol and more preferably still between 0.15 and 2.5 mol/mol.

Stage b)

According to stage b), the alumina support is brought into contact with at least one nickel metal salt, the melting point of said metal salt of which is between 20° C. and 150° C., for a period of time advantageously between 5 minutes and 5 hours, in order to form a solid mixture, the ratio by weight of said metal salt to the alumina support being between 0.1 and 2.3, preferably between 0.2 and 2.

Preferably, the metal salt is hydrated. Preferably, the metal salt is nickel nitrate hexahydrate (Ni(NO₃)₂.6H₂O, T_{melting}=56.7° C.).

According to stage b), the operation of bringing said porous oxide support and the nickel metal salt into contact can be carried out by any method known to a person skilled in the art. Preferably, use may be made of convective mixers, drum mixers or static mixers. Stage b) is advantageously carried out for a period of time of between 5 minutes and 5 hours, depending on the type of mixer used, preferably between 10 minutes and 4 hours.

In comparison with the prior art described in the document U.S. Pat. No. 5,036,032 and which is based on bringing a support into contact in a bath of molten salts, stage b) of the process according to the invention makes possible:
optimized control of the amount of metal deposited on the catalyst; and
controlled hazardousness and controlled cost of the preparation process by the minimization of the amounts of metal precursor employed, not exceeding 1 gram of metal precursor per 1 gram of support.

Implementation of Stages a) and b)

According to the invention:
stages a) and b) are carried out successively in this order, or
stages a) and b) are carried out simultaneously.

In a preferred embodiment, stage a) is carried out before carrying out stage b).

Stage c)

According to stage c), the mixture obtained on conclusion of stages a) and b) is heated with stirring to a temperature between the melting point of the metal salt and 200° C., and advantageously at atmospheric pressure. Preferably, the temperature is between 50 and 180° C. and more preferentially still between 60 and 160° C.

Advantageously, stage c) is carried out for a period of time of between 5 minutes and 12 hours, preferably between 5 minutes and 4 hours.

According to stage c), the mechanical homogenization of the mixture can be carried out by any method known to a person skilled in the art. Preferably, use may be made of convective mixers, drum mixers or static mixers. More preferentially still, stage c) is carried out by means of a drum mixer, the rotational speed of which is between 4 and 70 revolutions/minute, preferably between 10 and 60 revolutions/minute. This is because, if the rotation of the drum is too high, the active phase of the catalyst will not be distributed as a crust at the periphery of the support but will be distributed homogeneously throughout the support, which is not desirable.

Stage d) Catalyst Precursor Drying

Stage d) of drying the catalyst precursor obtained on conclusion of stage c) is carried out at a temperature of less than 250° C., preferably of between 15 and 180° C., more preferentially between 30 and 160° C., more preferentially still between 50 and 150° C. and in an even more preferential way between 70 and 140° C., typically for a period of time of between 10 minutes and 24 hours. Longer periods of time are not ruled out but do not necessarily contribute an improvement. The drying temperature of stage d) is generally higher than the heating temperature of stage c). Preferably, the drying temperature of stage d) is at least 10° C. higher than the heating temperature of stage c).

The drying stage can be carried out by any technique known to a person skilled in the art. It is advantageously carried out under an inert atmosphere or under an oxygen-containing atmosphere or under a mixture of inert gas and oxygen. It is advantageously carried out at atmospheric pressure or at reduced pressure. Preferably, this stage is carried out at atmospheric pressure and in the presence of air or nitrogen.

Stage e) Heat Treatment of the Dried Catalyst

The dried catalyst precursor undergoes an additional heat treatment stage, before the optional reduction stage f), at a temperature of between 250 and 1000° C. and preferably between 250 and 750° C., typically for a period of time of between 15 minutes and 10 hours, under an inert atmosphere or under an oxygen-containing atmosphere, in the presence or absence of water. Longer treatment times are not ruled out but do not necessarily contribute an improvement.

The term “heat treatment” is understood to mean the treatment in temperature respectively without the presence or in the presence of water. In the latter case, contact with steam can take place at atmospheric pressure or under autogenous pressure. Several combined cycles without the presence or with the presence of water can be carried out. After this or these treatment(s), the catalyst precursor comprises nickel in oxide form, that is to say in NiO form.

In the case of the presence of water, the water content is preferably between 150 and 900 grams per kilogram of dry air and more preferably still between 250 and 650 grams per kilogram of dry air.

Stacie f) Reduction by a Reducing Gas (Optional Stage)

Prior to the use of the catalyst in the catalytic reactor and the implementation of a hydrogenation process, at least one reducing treatment stage f) is advantageously carried out in the presence of a reducing gas after stage e), so as to obtain a catalyst comprising nickel at least partially in metallic form.

This treatment makes it possible to activate said catalyst and to form metal particles, in particular of nickel in the zero-valent state. Said reducing treatment can be carried out in situ or ex situ, that is to say after or before the catalyst is charged to the hydrogenation reactor.

The reducing gas is preferably hydrogen. The hydrogen can be used pure or as a mixture (for example a hydrogen/nitrogen or hydrogen/argon or hydrogen/methane mixture). In the case where the hydrogen is used as a mixture, all proportions can be envisaged.

Said reducing treatment is carried out at a temperature of between 120 and 500° C., preferably between 150 and 450° C. When the catalyst is not subjected to passivation or is subjected to a reducing treatment before passivation, the reducing treatment is carried out at a temperature of between 180 and 500° C., preferably between 200 and 450° C., and more preferentially still between 350 and 450° C. When the catalyst has been subjected beforehand to a passivation, the reducing treatment is generally carried out at a temperature of between 120 and 350° C., preferably between 150 and 350° C.

The duration of the reducing treatment is generally between 2 and 40 hours, preferably between 3 and 30 hours. The rise in temperature up to the desired reduction temperature is generally slow, for example set between 0.1 and 10° C./min, preferably between 0.3 and 7° C./min.

The hydrogen flow rate, expressed in I/hour/gram of catalyst, is between 0.01 and 100 I/hour/gram of catalyst, preferably between 0.05 and 10 I/hour/gram of catalyst and more preferably still between 0.1 and 5 I/hour/gram of catalyst.

Stage g) Passivation (Optional)

The catalyst prepared according to the process according to the invention can advantageously undergo a stage of passivation by a sulfur-containing compound which makes it possible to improve the selectivity of the catalysts and to prevent thermal runaways during the start-up of fresh catalysts. The passivation generally consists in irreversibly poisoning, by the sulfur-containing compound, the most virulent active sites of the nickel which exist on the fresh catalyst and in thus weakening the activity of the catalyst in favor of its selectivity. The passivation stage is carried out by the use of methods known to a person skilled in the art.

The stage of passivation by a sulfur-containing compound is generally carried out at a temperature of between 20 and 350° C., preferably between 40 and 200° C., for from 10 to 240 minutes. The sulfur-containing compound is, for example, chosen from the following compounds: thiophene, thiophane, alkyl monosulfides, such as dimethyl sulfide, diethyl sulfide, dipropyl sulfide and propyl methyl sulfide, or also an organic disulfide of formula HO—R₁—S—S—R₂—OH, such as dithiodiethanol of formula HO—C₂H₄—S—S—C₂H₄—OH (often referred to as DEODS). The sulfur content is generally between 0.1% and 2% by weight of said element, with respect to the total weight of the catalyst.

Process for the Hydrogenation of Aromatics

Another subject matter of the present invention is a process for the hydrogenation of at least one aromatic or polyaromatic compound contained in a hydrocarbon feedstock having a final boiling point of less than or equal to 650° C., generally between 20 and 650° C. and preferably between 20 and 450° C. Said feedstock of hydrocarbons containing at least one aromatic or polyaromatic compound can be chosen from the following petroleum or petrochemical fractions: reformate from catalytic reforming, kerosene, light gas oil, heavy gas oil, cracking distillates, such as fluid catalytic cracking (FCC) cycle oil, gas oil from coking units or hydrocracking distillates.

The content of aromatic or polyaromatic compounds contained in the hydrocarbon feedstock treated in the hydrogenation process according to the invention is generally between 0.1% and 80% by weight, preferably between 1% and 50% by weight and particularly preferably between 2% and 35% by weight, the percentage being based on the total weight of the hydrocarbon feedstock. The aromatic compounds present in said hydrocarbon feedstock are, for example, benzene or alkylaromatics, such as toluene, ethylbenzene, o-xylene, m-xylene or p-xylene, or also aromatics having several aromatic rings (polyaromatics), such as naphthalene.

The sulfur or chlorine content of the feedstock is generally less than 5000 ppm by weight of sulfur or chlorine, preferably less than 100 ppm by weight and particularly preferably less than 10 ppm by weight.

The technological implementation of the process for the hydrogenation of aromatic or polyaromatic compounds is, for example, carried out by injection, as upflow or downflow, of the hydrocarbon feedstock and of the hydrogen into at least one fixed bed reactor. Said reactor can be of isothermal type or of adiabatic type. An adiabatic reactor is preferred. The hydrocarbon feedstock can advantageously be diluted by one or more reinjection(s) of the effluent, resulting from said reactor where the reaction for the hydrogenation of the aromatics takes place, at various points of the reactor, located between the inlet and the outlet of the reactor, in order to limit the temperature gradient in the reactor. The technological implementation of the process for the hydrogenation of the aromatics according to the invention can also advantageously be carried out by the implantation of at least said supported catalyst in a reactive distillation column or in reactors-exchangers or in a reactor in which the catalyst is in suspension (slurry). The stream of hydrogen can be introduced at the same time as the feedstock to be hydrogenated and/or at one or more different points of the reactor.

The hydrogenation of the aromatic or polyaromatic compounds can be carried out in the gas phase or in the liquid phase, preferably in the liquid phase. Generally, the hydrogenation of the aromatic or polyaromatic compounds is carried out at a temperature of between 30 and 350° C., preferably between 50 and 325° C., at a pressure of between 0.1 and 20 MPa, preferably between 0.5 and 10 MPa, at a hydrogen/(aromatic compounds to be hydrogenated) molar ratio between 0.1 and 10 and at an hourly space velocity HSV of between 0.05 and 50 h⁻¹, preferably between 0.1 and 10 h⁻¹, of a hydrocarbon feedstock containing aromatic or polyaromatic compounds and having a final boiling point of less than or equal to 650° C., generally between 20 and 650° C. and preferably between 20 and 450° C.

The hydrogen flow rate is adjusted in order to have available a sufficient amount thereof to theoretically hydrogenate all of the aromatic compounds and to maintain an excess of hydrogen at the reactor outlet.

The conversion of the aromatic or polyaromatic compounds is generally greater than 20 mol %, preferably greater than 40 mol %, more preferably greater than 80 mol % and particularly preferably greater than 90 mol % of the aromatic or polyaromatic compounds contained in the hydrocarbon feedstock. The conversion is calculated by dividing the difference between the total moles of the aromatic or polyaromatic compounds in the hydrocarbon feedstock and in the product by the total moles of the aromatic or polyaromatic compounds in the hydrocarbon feedstock.

According to a specific alternative form of the process according to the invention, a process for the hydrogenation of the benzene of a hydrocarbon feedstock, such as the reformate resulting from a catalytic reforming unit, is carried out. The benzene content in said hydrocarbon feedstock is generally between 0.1% and 40% by weight, preferably between 0.5% and 35% by weight and particularly preferably between 2% and 30% by weight, the percentage by weight being based on the total weight of the hydrocarbon feedstock.

The sulfur or chlorine content of the feedstock is generally less than 10 ppm by weight of sulfur or chlorine respectively and preferably less than 2 ppm by weight.

The hydrogenation of the benzene contained in the hydrocarbon feedstock can be carried out in the gas phase or in the liquid phase, preferably in the liquid phase. When it is carried out in the liquid phase, a solvent can be present, such as cyclohexane, heptane or octane. Generally, the hydrogenation of the benzene is carried out at a temperature of between 30 and 250° C., preferably between 50 and 200° C. and more preferably between 80 and 180° C., at a pressure of between 0.1 and 10 MPa, preferably between 0.5 and 4 MPa, at a hydrogen/(benzene) molar ratio between 0.1 and 10 and at an hourly space velocity HSV of between 0.05 and 50 h⁻¹, preferably between 0.5 and 10 h⁻¹.

The conversion of the benzene is generally greater than 50 mol %, preferably greater than 80 mol %, more preferably greater than 90 mol % and particularly preferably greater than 98 mol %.

The invention will now be illustrated via the examples below which are in no way limiting.

### EXAMPLES

For all the catalysts mentioned in the examples mentioned below, the support is an alumina AL-1 exhibiting a specific surface of 80 m²/g, a pore volume of 0.7 ml/g and a median mesopore diameter of 12 nm.

### Example 1 (Conforms)

10 g of alumina AL-1 support are brought into contact with 1.96 g of citric acid dissolved in 5.4 g of water. The solid thus obtained is subsequently dried in an oven at 60° C. for 2 hours and then at 120° C. for 12 hours.

Subsequently, the support is brought into contact with 9.47 g of nickel nitrate hexahydrate in a pan at 25° C. which rotates at a speed of 40 to 50 revolutions per minute. The pan is subsequently heated to 62° C. and rotates at a speed of 40 to 50 revolutions per minute for 15 minutes. The molar ratio by weight of the citric acid to the nickel is 0.2.

The nickel content targeted with regard to this stage is 25% by weight of Ni, with respect to the weight of the final catalyst. The solid thus obtained is subsequently dried in an oven at 120° C. overnight and then calcined under a stream of air of 1 I/h/g of catalyst at 450° C. for 2 hours.

The catalyst A containing 28% by weight of the element nickel, with respect to the total weight of the catalyst, is obtained. The characteristics of the catalyst A thus obtained are given in table 1 below.

### Example 2 (Conforms)

10 g of alumina AL-1 support are brought into contact with 3.96 g of citric acid dissolved in 10 g of water. The solid thus obtained is subsequently dried in an oven at 60° C. for 2 hours and then at 120° C. for 12 hours. Subsequently, the support is brought into contact with 9.47 g of nickel nitrate hexahydrate in a pan at 25° C. which rotates at a speed of 40 to 50 revolutions per minute. The pan is subsequently heated to 62° C. and rotates at a speed of 40 to 50 revolutions per minute for 15 minutes. The citric acid to Ni molar ratio is 0.4.

The nickel content targeted with regard to this stage is 25% by weight of Ni, with respect to the weight of the final catalyst. The solid thus obtained is subsequently dried in an oven at 120° C. overnight and then calcined under a stream of air of 1 I/h/g of catalyst at 450° C. for 2 hours.

The catalyst B containing 25% by weight of the element nickel, with respect to the total weight of the catalyst, is obtained. The characteristics of the catalyst B thus obtained are given in table 1 below.

### Example 3 (Conforms)

10 g of alumina AL-1 support are brought into contact with 0.77 g of glycolic acid dissolved in 5.4 g of water. The solid thus obtained is subsequently dried in an oven at 60° C. for 2 hours and then at 120° C. for 12 hours.

Subsequently, the support is brought into contact with 9.47 g of nickel nitrate hexahydrate in a pan at 25° C. which rotates at a speed of 40 to 50 revolutions per minute. The pan is subsequently heated to 62° C. and rotates at a speed of 40 to 50 revolutions per minute for 15 minutes. The glycolic acid to Ni molar ratio is 0.2.

The Ni content targeted with regard to this stage is 25% by weight of Ni, with respect to the weight of the final catalyst. The solid thus obtained is subsequently dried in an oven at 120° C. overnight and then calcined under a stream of air of 1 I/h/g of catalyst at 450° C. for 2 hours.

The catalyst C containing 25% by weight of the element nickel, with respect to the total weight of the catalyst, is obtained. The characteristics of the catalyst C thus obtained are given in table 1 below.

### Example 4 (not in Accordance)

10 g of alumina AL-1 support are dry impregnated with 15.78 g of nickel nitrate hexahydrate in a pan at 25° C. which rotates at a speed of 40 to 50 revolutions per minute. The pan is subsequently heated to 62° C. and rotates at a speed of 40 to 50 revolutions per minute for 15 minutes.

The Ni content targeted with regard to this stage is 25% by weight of Ni, with respect to the weight of the final catalyst. The solid thus obtained is subsequently dried in an oven at 120° C. overnight and then calcined under a stream of air of 1 I/h/g of catalyst at 450° C. for 2 hours.

The catalyst D containing 25% by weight of the element nickel, with respect to the total weight of the catalyst, is obtained. The characteristics of the catalyst D thus obtained are given in table 1 below.

### Example 5: Characterization

All the catalysts contain the contents targeted during the impregnation, that is to say 25% (characterized by X-ray fluorescence), with respect to the total weight of the catalyst. The sizes of NiO particles obtained after the calcination stage was determined by X-ray diffraction (XRD) analysis on samples of catalyst in powder form. The characteristics of the catalysts A to D are listed in table 1 below.

| | Ni content | Size of the particles |
|---|---|---|
| Catalyst | (wt %) | (nm) |
| A (in accordance) | 25 | 2.8 |
| B (in accordance) | 25 | 2.3 |
| C (in accordance) | 25 | 2.8 |
| D (not in accordance) | 25 | 21 |

### Example 6: The Catalysts A to D Described in the Examples Above are Tested with Regard to the Reaction for the Hydrogenation of Toluene

The reaction for the hydrogenation of toluene is carried out in a 500 ml stainless steel autoclave which is provided with a magnetically-driven mechanical stirrer and which is able to operate under a maximum pressure of 100 bar (10 MPa) and temperatures of between 5° C. and 200° C.

Prior to its introduction into the autoclave, an amount of 2 ml of catalyst is reduced ex situ under a stream of hydrogen of 1 I/h/g of catalyst at 400° C. for 16 hours (temperature rise gradient of 1° C./min) and then it is transferred into the autoclave, with the exclusion of air. After addition of 216 ml of n-heptane (supplied by VWR®, purity >99% Chromanorm HPLC), the autoclave is closed, purged, then pressurized under 35 bar (3.5 MPa) of hydrogen and brought to the temperature of the test, which is equal to 80° C. At the time t=0, approximately 26 g of toluene (supplied by SDS®, purity >99.8%) are introduced into the autoclave (the initial composition of the reaction mixture is then toluene 6% by weight/n-heptane 94% by weight) and stirring is started at 1600 rev/min. The pressure is kept constant at 35 bar (3.5 MPa) in the autoclave using a storage cylinder located upstream of the reactor.

The progress of the reaction is monitored by taking samples from the reaction medium at regular time intervals: the toluene is completely hydrogenated to give methylcyclohexane. The hydrogen consumption is also monitored over time by the decrease in pressure in a storage cylinder located upstream of the reactor. The catalytic activity is expressed in moles of H₂ consumed per minute and per gram of Ni.

The catalytic activities measured for the catalysts A to D are given in table 2 below. They are with reference to the catalytic activity measured for the catalyst D (A_{HYD}).

| | | Size of the | |
|---|---|---|---|
| | Ni content | Ni° particles | A_{HYDRAro} |
| Catalyst | (%) | (nm) | (%) |
| A (in accordance) | 25 | 2.8 | 220 |
| B (in accordance) | 25 | 2.3 | 250 |
| C (in accordance) | 25 | 2.8 | 250 |
| D (not in accordance) | 25 | 21 | 100 |

The catalysts A, B and C according to the invention result in very high selective hydrogenation activities. In example 4, the additive was not added, which results in the catalyst D with a greatly reduced activity due to the size of the nickel particles of 20 nm, i.e. 10 times greater than for the catalysts according to the invention.

1. A catalyst for the hydrogenation of aromatic or polyaromatic compounds comprising a nickel-based active phase and an alumina support, said active phase does not comprising a metal from Group VIB, said catalyst comprising between 20% and 60% by weight of elemental nickel, with respect to the total weight of the catalyst, the size of the nickel particles in the catalyst, measured in oxide form, being less than 18 nm, said catalyst being capable of being obtained by the process comprising at least the following stages:
a) the alumina support is brought into contact with at least one organic additive comprising oxygen and/or nitrogen, the molar ratio of the organic additive to the nickel being greater than 0.05 mol/mol;
b) the alumina support is brought into contact with at least one nickel metal salt, at a temperature of less than the melting point of said nickel metal salt, in order to form a solid mixture, the ratio by weight of said metal salt to the alumina support being between 0.1 and 2.3,
stages a) and b) being carried out either successively in this order, or simultaneously;
c) the solid mixture obtained on conclusion of stages a) and b) is heated with stirring to a temperature between the melting point of said metal salt and 200° C., in order to obtain a catalyst precursor;
d) the catalyst precursor on conclusion of stage c) is dried at a temperature of less than 250° C. in order to obtain a dried catalyst precursor;
e) a stage of heat treatment of the dried catalyst precursor obtained on conclusion of stage d) is carried out at a temperature of between 250 and 1000° C.

2. The catalyst as claimed in claim 1, characterized in that the size of the nickel particles in the catalyst, measured in oxide form, is between 0.5 and 12 nm.

3. The catalyst as claimed in claim 1, characterized in that the size of the nickel particles in the catalyst, measured in oxide form, is between 1 and 5 nm.

4. A process for the preparation of a catalyst for the hydrogenation of aromatic or polyaromatic compounds comprising a nickel-based active phase and an alumina support, said active phase does not comprising a metal from Group VIB, said catalyst comprising between 20% and 60% by weight of elemental nickel, with respect to the total weight of the catalyst, the size of the nickel particles in the catalyst, measured in oxide form, being less than 18 nm, said process comprising the following stages:
a) the alumina support is brought into contact with at least one organic additive comprising oxygen and/or nitrogen, the molar ratio of the organic additive to the nickel being greater than 0.05 mol/mol;
b) the alumina support is brought into contact with at least one nickel metal salt, at a temperature of less than the melting point of said nickel metal salt, in order to form a solid mixture, the ratio by weight of said metal salt to the alumina support being between 0.1 and 2.3,
stages a) and b) being carried out either successively in this order, or simultaneously;
c) the solid mixture obtained on conclusion of stages a) and b) is heated with stirring to a temperature between the melting point of said metal salt and 200° C., in order to obtain a catalyst precursor;
d) the catalyst precursor obtained on conclusion of stage c) is dried at a temperature of less than 250° C. in order to obtain a dried catalyst precursor;
e) a stage of heat treatment of the dried catalyst precursor obtained on conclusion of stage d) is carried out at a temperature of between 250 and 1000° C.

5. The process as claimed in claim 4, in which the melting point of said metal salt is between 20° C. and 150° C.

6. The process as claimed in claim 4, in which the molar ratio of said organic additive introduced in stage a) to the element nickel introduced in stage b) is between 0.1 and 5.0 mol/mol.

7. The process as claimed in claim 4, in which stages a) and b) are carried out simultaneously.

8. The process as claimed in claim 4, in which the organic additive is chosen from aldehydes including from 1 to 14 carbon atoms per molecule, ketones or polyketones including from 3 to 18 carbon atoms per molecule, ethers and esters including from 2 to 14 carbon atoms per molecule, alcohols or polyalcohols including from 1 to 14 carbon atoms per molecule and carboxylic acids or polycarboxylic acids including from 1 to 14 carbon atoms per molecule, or a combination of the various functional groups above.

9. The process as claimed in claim 4, in which said organic additive of stage a) is chosen from formic acid, formaldehyde, acetic acid, citric acid, oxalic acid, glycolic acid, malonic acid, levulinic acid, ethanol, methanol, ethyl formate, methyl formate, paraldehyde, acetaldehyde, γ-valerolactone, glucose and sorbitol.

10. The process as claimed in claim 9, in which the organic additive is chosen from citric acid, formic acid, glycolic acid, levulinic acid and oxalic acid.

11. The process as claimed in claim 4, in which stage c) is carried out by means of a pan operating at a speed of between 4 and 70 revolutions per minute.

12. The process as claimed in claim 4, in which, in stage b), the ratio by weight of said metal salt to the alumina support is between 0.2 and 2.

13. A process for the hydrogenation of at least one aromatic or polyaromatic compound present in a hydrocarbon feedstock having a final boiling point of less than or equal to 650° C., said process being carried out in the gas phase or in the liquid phase, at a temperature of between 30 and 350° C., at a pressure of between 0.1 and 20 MPa, at a hydrogen/(aromatic compounds to be hydrogenated) molar ratio between 0.1 and 10 and at an hourly space velocity HSV of between 0.05 and 50 h⁻¹, in the presence of a catalyst as claimed in claim 1.
